(19) United States
(12) Patent Application Publication
(10) Pub. No.: US 2024/0350378 A1
(43) Pub. Date: Oct. 24, 2024

(54) DEODORANT COSMETIC PRODUCT

(71) Applicant: Joban Beauty, Inc., New York, NY (US)

(72) Inventors: Sharanjit Kaur Deol, New York, NY (US); Ron Robinson, New York, NY (US); Peter Bohm, South Plainfield, NJ (US)

(73) Assignee: Joban Beauty, Inc., New York, NY (US)

(21) Appl. No.: 18/396,117

(22) Filed: Dec. 26, 2023

Related U.S. Application Data
(63) Continuation of application No. 16/855,697, filed on Apr. 22, 2020, now abandoned.
(60) Provisional application No. 62/914,001, filed on Oct. 11, 2019.
(60) Provisional application No. 62/879,503, filed on Jul. 28, 2019.
(60) Provisional application No. 62/836,808, filed on Apr. 22, 2019.

(51) Int. Cl. A61K 8/19; A45D 34/04; A61K 8/25; A61K 8/92; A61K 8/96; A61K 8/9789; A61K 8/9794; A61Q 15/00

(52) U.S. Cl. CPC A61K 8/19; A45D 34/04; A61K 8/25; A61K 8/922; A61K 8/925; A61K 8/927; A61K 8/965; A61K 8/9789; A61K 8/9794; A61Q 15/00; A45D 2200/056; A45D 2200/1009

(57) ABSTRACT

A personal care composition that can serve as a deodorant and optionally, as a concealer, a colorant, and/or a brightener. The personal care composition can include: (i) a deodorant agent in an amount effective to reduce sweat and/or body odor; and (ii) a film former. Optionally, the personal care composition can further include a pigment. The personal care composition can be characterized by substantial non-transferability, and can serve to reduce sweat and/or body order, while also concealing discoloration, imperfections, dark spots, large pores and darkening, for example, in the underarm.

### RELATED APPLICATIONS

This application is a continuation application of U.S. patent application Ser. No. 16/855,697, filed Apr. 22, 2020, which claims priority to U.S. Provisional Application No. 62/836,808, filed Apr. 22, 2019, U.S. Provisional Application No. 62/879,503, filed Jul. 28, 2019, and U.S. Provisional Application No. 62/914,001, filed Oct. 11, 2019. The entire contents of each of these applications are hereby incorporated by reference herein.

### BACKGROUND

Underarms are often characterized by skin discoloration, imperfections and darkening. While individuals often attempt to use makeup to address these issues, this often results in makeup clogging pores, and increased sweat and body odor. Moreover, chemicals in strong deodorants often exacerbate the discoloration and darkening of underarms.

Accordingly, there is a need for a cosmetic preparation that can both address underarm discoloration and darkening and reduce body odor.

### SUMMARY

The present invention is generally directed to a cosmetic preparation that exhibits deodorant properties and, optionally, concealer or brightening properties. In a first aspect, the present invention is directed to a personal care composition that reduces body odor including at least one of sodium bicarbonate, arrowroot, cornstarch, bentonite clay, kaolin clay, activated charcoal powder, tapioca starch, and magnesium hydroxide in an amount effective to reduce sweat and/or body odor, wherein the composition conceals skin discoloration, imperfections, dark spots, large pores or darkening.

In certain embodiments, the composition includes sodium bicarbonate and, optionally, cornstarch. In addition, where the composition includes sodium bicarbonate, the composition can further include arrowroot.

Alternatively, or in combination, the personal composition includes kaolin clay and/or bentonite clay.

In one embodiment, the personal care composition includes tapioca starch. In one embodiment, the personal care composition includes sodium bicarbonate and tapioca starch.

In various embodiments, the personal care composition includes a spreadability agent selected from the group consisting of isododecane, trisiloxane, cyclopentasiloxane, a trisiloxane blend and a combination thereof. In a particular embodiment, the composition includes isododecane.

In various embodiments, the personal care composition further includes a thickening agent. In various embodiments, the thickening agent may be a wax, a montmorillonite mineral, a quaternized derivative of a montmorillonite mineral or a combination thereof.

For example, the wax may be acacia, beeswax, ceresin, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG 6-20 beeswax, PEG-12 carnauba wax, and a combination thereof. In certain embodiments, the thickening agent is a silicone wax including a silicone selected from the group consisting of stearoxydimethicone, behenoxy dimethicone, stearyl dimethicone, cetearyl dimethicone, and a combination thereof. Alternatively or in combination, the thickening agent is a montmorillonite mineral comprising hectorite or bentonite. In a particular embodiment, the thickening agent is bentonite. In an alternative embodiment, the thickening agent is disteardimoium hectorite.

In further embodiments, the personal care composition includes a preservative. For example, the preservative may be benzyl alcohol, propylparaben and a combination thereof.

In further embodiments, the personal care composition includes a water resistant agent. In certain embodiments, the water resistant agent may be a silica polymer, for example, silica silylate, silica dimethyl silylate, trimethylsiloxysilicate, trifluoropropyldimethyl/trimethylsiloxysilicate, or a combination thereof. In a particular embodiment, the water resistant agent includes trimethylsiloxysilicate. Alternatively or in combination, the water resistant agent includes beeswax.

In certain embodiments, the water resistant agent serves as a film former, for example, trimethylsiloxysilicate. Alternatively, or in addition, the composition may include a film former, for example, trimethylsiloxysilicate.

In further embodiments, the personal care composition includes a fragrance agent, for example, a natural fragrance powder. By way of example, the fragrance agent may have a scent selected from the group consisting of tea tree, lavender, lemongrass, citrus, rosemary, and a combination thereof.

In yet another embodiment, the personal care composition includes a fragrance absorption agent, for example, dextrose.

In further embodiments, the personal care composition is substantially free of aluminum. Alternatively or in combination, the composition may be substantially free of a paraben. Alternatively or in combination, the composition may be substantially free of propylene glycol.

In yet another embodiment, the personal care composition includes a colorant. In a particular embodiment, the colorant is a pigment. For example, the pigment may be selected from the group consisting of titanium dioxide, yellow iron oxide, red iron oxide, black iron oxide, and combinations thereof.

In particular embodiments, the personal care composition includes isododecane and either (a) sodium bicarbonate and arrowroot, (b) sodium bicarbonate, cornstarch and arrowroot, (c) cornstarch, (d) bentonite clay, or (e) kaolin clay. In a particular embodiment, the composition includes isododecane, sodium bicarbonate and arrowroot. The composition may further include either (f) trimethylsiloxysilicate, disteardimoium hectorite and propylparaben; or (g) beeswax, bentonite and benzyl alcohol. The composition may further include a fragrance agent and dextrose.

In a particular embodiment, the personal care composition includes sodium bicarbonate and isododecane. The composition may further include tapioca starch. In a particular embodiment, the composition further includes trimethylsiloxysilicate. In yet a further embodiment, the composition further includes a pigment, for example, a pigment selected from the group consisting of titanium dioxide, yellow iron oxide, red iron oxide, black iron oxide, and combinations thereof.

In a particular preferable embodiment of the foregoing aspect, the personal care composition of the foregoing embodiment is substantially non-transferable. In a particular embodiment of the foregoing aspect, the personal care composition absorbs sweat. Alternatively or in combination, the personal care composition adsorbs sweat.

In a second aspect, the present invention provides a personal care composition that reduces body odor including: (i) a deodorant agent in an amount effective to reduce sweat and/or body odor; and (ii) a film former.

In certain embodiments of the foregoing aspect, the personal care composition includes: (i) a deodorant agent in an amount effective to reduce sweat and/or body odor; (ii) a film former; and (iii) a pigment.

In certain embodiments of the foregoing aspect, the personal care composition includes: (i) a deodorant agent in an amount effective to reduce sweat and/or body odor; (ii) a film former; and (iii) a brightening agent.

In certain embodiments of the foregoing aspect, the personal care composition includes: (i) a deodorant agent in an amount effective to reduce sweat and/or body odor; (ii) a film former; (iii) a pigment; and (iv) a brightening agent.

In a particular preferable embodiment of the second aspect, the personal care composition of the foregoing embodiment is substantially non-transferable. In a particular embodiment of the foregoing aspect, the personal care composition absorbs sweat. Alternatively or in combination, the personal care composition adsorbs sweat.

In certain embodiments, the personal care composition further includes a volatile spreadability agent.

In certain embodiments of the foregoing aspect, the deodorant agent includes at least one of sodium bicarbonate, arrowroot, cornstarch, bentonite clay, kaolin clay, activated charcoal powder, tapioca starch, and magnesium hydroxide in an amount effective to reduce sweat and/or body odor. In a particular embodiment, the deodorant agent includes sodium bicarbonate, tapioca starch or a combination thereof.

In certain embodiments of the foregoing aspect of the invention, the film former is selected from the group consisting of trimethylsiloxysilicate, a polymethylsilsesquioxane, a tosylamide/formaldehyde Resin, a polyester resin, a trimellitic anhydride monomer, a vinyl caprolactam/VP/dimethylaminoethyl methacrylate copolymer, isododecane and ethylene/propylene copolymer, styrene/butadiene copolymer, polyvinylpyrrolidone (PVP), a PVP and VP/cicosene copolymer, triacontanyl PVP, a styrene/acrylates copolymer, a VP/Eicosene Copolymer, an acrylates copolymers, an acrylates/C12-22 alkyl methacrylate copolymer, VP/Dimethiconylacrylate/Polycarbamyl Polyglycol Ester (and) VP/Polycarbamyl Polyglycol Ester, VP/Polycarbamyl, Adipic Acid/Neopentyl Glycol/Trimellitic Anhydride Copolymer, Polyurethane-18,48 and alike, Polyester Resins, a Vinyl Caprolactam/VP/Dimethylaminoethyl Methacrylate copolymer, Maltose (and) Agave Tequilana Leaf Extract, an Ethylene/Propylene copolymer, a styrene/butadiene copolymer, a soyamine/IPDI copolymer, trifluoropropyldimethylsiloxy/trimethylsiloxy Silsesquioxane (and) dimethicone.

In a particular embodiment of the foregoing aspect of the invention, the personal care composition is an oil based composition. In such embodiment, the film former may be selected from the group consisting of trimethylsiloxysilicate, a polymethylsilsesquioxane, a tosylamide/formaldehyde Resin, a polyester resin, a trimellitic anhydride monomer, a vinyl caprolactam/VP/dimethylaminoethyl methacrylate copolymer, isododecane and ethylene/propylene copolymer, styrene/butadiene copolymer, polyvinylpyrrolidone (PVP), a PVP and VP/cicosene copolymer, triacontanyl PVP and a combination thereof. In a particular embodiment, the film former is trimethylsiloxysilicate.

In an alternative embodiment of the foregoing aspect of the invention, the personal care composition is a water based composition. In such embodiment, the film former may be selected from the group consisting of an acrylates copolymer, an acrylates/octylacrylamide copolymer, a styrene/acrylates copolymer, polyvinyl alcohol (PVA), and a combination thereof. In a particular embodiment, the film former is a styrene/acrylates copolymer.

The personal care composition of the foregoing invention may further include a pigment, for example, selected from the group consisting of titanium dioxide, yellow iron oxide, red iron oxide, black iron oxide, and combinations thereof.

In various embodiments of the foregoing aspect of the invention, the composition is an emulsion. The emulsion may include an emulsifier, e.g., a nonionic, anionic, cationic or zwitterionic emulsifier. Exemplary emulsifiers for use in the present invention include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

In a particular embodiment of the foregoing aspect, the personal care composition is water based and the emulsifier comprises at least one of butylene glycol, cetearyl alcohol, polysorbate 60, propylene glycol stearate, and a combination thereof. In another particular embodiment, the composition is water based and the emulsifier has an HLB value between 8 and 18.

In an alternative embodiment, the composition is an oil based emulsion, wherein water forms the inner phase of the emulsion. In such embodiments, the composition may include an emulsifier having an HLB value between 4 and 6.

Emulsions may also be formed by stirring, high shear mixing, high-pressure homogenization or high-amplitude ultrasound (sonication). Optionally, such emulsions are substantially free of an emulsifier.

Alternatively, the personal care composition of the foregoing aspect of the invention is anhydrous.

In certain embodiments, the personal care compositions of the present invention include a hyper-pigmentation treatment agent, for example, mulberry (e.g., mulberry extract), licorice, kojic acid, Vitamin B3 (niacinamide), arbutin, tyrosine inhibitor or a combination thereof.

In certain embodiments, the personal care compositions of the present invention further includes a brightening agent, for example, licorice, kojic acid, arbutrin, mulberry (e.g., mulberry extract), Vitamin B3 (niacinamide), Vitamin C, citrus extracts, willow bark extract, lactic acid, Aloe vera, bearberry extract, Phyllanthus emblica, ALPAFLOR® GIGAWHITE™ (INCI name: Water, Glycerin, Alcohol, Malva sylvestris (Mallow) Extract, Mentha piperita (Peppermint) Leaf Extract, Primula Veris Extract, Alchemilla Vulgaris Extract, Veronica Officinalis Extract, Melissa Officinalis Leaf Extract, Achillea Millefolium Extract), glycolic acid, phenylethyl resorcinol, bisabolol, butyl methoxydibenzoylmethane, 4-butylresorcinol, Pelvetia canaliculata extract, synthetic fluorphlogopite, titanium dioxide, tin oxide, Hexapeptide-2, Poncirus trifoliata fruit extract, 1-Methylhydantoin-2-Imide, Bambusa arundinacea stem extract, Equisetum fluviatile extract, Camellia Japonica flower extract, Pinus densiflora leaf extract, Terminalia ferdinandiana fruit extract, Carica papaya (papaya) fruit extract, Arctostaphylos uva-ursi leaf extract, or a combination thereof. In some embodiments, the brightening agent is capable of inhibiting melanin synthesis. In some embodiments, the brightening agent has anti-tyrosinase activity.

In some embodiments, certain agents, for example, mulberry (e.g., mulberry extract), licorice, kojic acid, Vitamin B3 (niacinamide), arbutin or a combination thereof can serve as both a hyper-pigmentation treatment agent and a brightening agent.

In another aspect, the present invention is directed to a cosmetic product including the personal care composition, as described herein, contained within a device. The device may include an applicator for applying the composition to the body, optionally, the underarm. Preferably, the applicator is a foam or sponge. In addition, the device may further include a base which holds the personal care composition. Further, the device may include a pump. In alternative embodiments, the device is an airbrush or aerosol spray.

In a further aspect, the present invention is directed to a method of treating a subject suffering from hyper-pigmentation by applying the personal care compositions of the invention, in particular, those compositions including a hyper-pigmentation treatment agent. For example, the personal care composition is applied at least once a day.

### DETAILED DESCRIPTION

The present application is directed to deodorant compositions and, in particular, a non-transferable deodorant composition. In one aspect, the present application is directed to a personal care composition that serves to reduce sweat and/or body order, including a deodorant agent and a film former. In a particular embodiment, the personal care composition serves to absorb and/or adsorb sweat.

In another aspect, the present application is directed to a dual use personal care composition that serves as both a deodorant and a concealer. In another aspect, the present application provides a tri-use personal care composition that serves as a deodorant, a concealer and to color correct. The present invention addresses a significant need in the cosmetic industry for a product that serves to reduce body odor, while also concealing discoloration, imperfections, dark spots, large pores and darkening, for example, in the underarm.

Developing a composition that can serve both purposes presents considerable challenges. For the composition to effectively serve its multiple purposes, it should be non-transferable, water resistant, sweat proof, prevent odor, provide a fragrant smell and provide full cosmetic coverage. Achieving each of these functions without sacrificing, for example, the deodorant function or alternatively the concealing function, presents considerable challenges. Additionally, achieving each of these functions, while also serving to correct color, presents even further challenges. For example, allowing for the transfer of the deodorant agent and the concealer, while minimizing or preventing transfer of the pigment presents a considerable challenge.

As used herein, reference to the ability of the composition to minimize or prevent the transfer of the composition refers to the quality of the composition to be substantially resistant to the transfer of the composition itself and/or color (e.g., pigment) upon contact with objects, such as clothing. The degree of transferability can be assessed by art known techniques, for example, as disclosed in U.S. Pat. No. 6,406,683, the entire contents of which are incorporated herein by reference.

Moreover, finding a form for the composition presents yet even further challenges in that the two standard delivery mechanisms for deodorants may be unsuitable for the desired functions. Specifically, deodorant sticks can often be too messy and require additional steps to blend and apply the formulation as desired. Alternatively, deodorant sprays are not suitable to target a particular area, for example, of discoloration, and also require additional steps to blend and apply the formulation as desired.

Preferably, the compositions of the invention are in liquid form. The consistency of the composition is preferably consistent with that of a cream to allow for case of spreadability and topical application for targeted areas. The compositions may be either an anhydrous composition or a water based composition.

Achieving the foregoing properties, e.g., combining the function of a deodorant as well as a pigmented concealer to cover uneven and unattractive hyper and hypo pigmentation in the under-arm area to achieve an even skin tone yet remain non-transferable, required the incorporation of a film forming system aimed at affixing product onto the affected area which is typical of a higher moisture content and high friction activity between the skin's surface and contact with clothing. A significant challenge was presented to maintain adhesion onto the skin and at the same time prevent transferring of the product and pigment onto clothing without the film breaking down over extended time of wear, despite exposure to moisture and rubbing friction. Quite significantly, the present invention provides systems that function equally in either water or oil based formulations. Finally, the formulations provided herein are of a rheology which functions well with either an extruded type of package application, air tight stick, and airbrush applications without changing the essential properties as discussed above.

Preferably, the compositions of the invention are free of aluminum based products, parabens, and/or propylene glycol, common ingredients in cosmetic products that may be associated with certain health risks, for example, cancer. In particular, aluminum, which is commonly found in deodorants and anti-perspirants, may be associated with cancer and darkening of the skin, one of the underlying problems the present invention is directed to addressing. For example, some studies have indicated that use of aluminum based compounds may be a risk factor for the development of breast cancer or Alzheimer's Disease.

All formulation/ingredient percentages are by weight, based on the weight of the composition, unless otherwise specified. All ratios are weight ratios, unless specifically stated otherwise. All numeric ranges are inclusive of narrower ranges; delineated upper and lower range limits are interchangeable to create further ranges not explicitly delineated. The number of significant digits conveys neither limitation on the indicated amounts nor on the accuracy of the measurements.

### Compositions

The compositions of the invention may be low viscosity aqueous, alcoholic, or hydro-alcoholic suspensions. Alternatively, the compositions may be aqueous, alcoholic, or hydro-alcoholic gels. Alternative compositions include stick form, cream forms, airbrush, aerosols as well as powders with fixatives.

Specifically, the personal care compositions of the present invention may be a water based or an oil based composition.

### (i) Water Based Compositions

In certain embodiments, the compositions are a water based composition. In a particular embodiment, the water based composition is an emulsion, in which water constitutes the outer phase. Further oil based elements, for example, including emollients and pigments, may be present in the inner phase of the emulsion. Alternatively, or in combination, certain elements, for example, pigments, may be present in a middle phase of the emulsion. The evaporation of the water in the outer phase of the emulsion leaves behind the film as well as the non-volatile components of the oil phase, e.g., essential oils and the remaining solids and oils.

In some embodiments, the brightening agents described herein may be water soluble or oil soluble. Accordingly, within the context of a water based composition the brightening agents may be found in any of the outer phase, middle phase, and/or inner phase depending on the particular solubility properties of the individual brightening agent.

In a particular embodiment, the water based emulsion includes an emulsifier. For example, the emulsifier may be nonionic, anionic, cationic or zwitterionic. The emulsifier preferably has an HLB value between 8-18. Exemplary emulsions for use in the compositions of the invention are set forth below.

In another embodiment, the emulsion may be formed by stirring, high shear mixing, high-pressure homogenization or high-amplitude ultrasound (sonication). Optionally, in such embodiments, the composition is substantially free of an emulsifier, as the emulsion is formed by such techniques.

### (ii) Oil Based Compositions

Alternatively, the compositions of the present invention may be oil based compositions. In one embodiment, the oil based composition is a nonaqueous formulation. Alternatively, the oil based composition is an emulsion.

In exemplary embodiments where the oil based composition is an emulsion, water constitutes the inner phase and the oil and volatile oils (e.g., isododecane or cyclomethicones) are in the outer phase. The outer phase suspends the pigments. Upon evaporation of the volatile oils, the oil based film former, e.g., trimethylsiloxysilicate, remains along with the now coated pigments which have become part of the film.

In some embodiments, the brightening agents described herein may be water soluble or oil soluble. Accordingly, within the context of an oil based composition, the brightening agents may be found in either the outer phase and/or inner phase depending on the particular solubility properties of the individual brightening agent. Additionally, in some embodiments, the brightening agent may be heat sensitive and be added to the oil based composition at room temperature or under cooling conditions.

In a particular embodiment, the oil based emulsion includes an emulsifier. For example, the emulsifier may be nonionic, anionic, cationic or zwitterionic. The emulsifier preferably has an HLB value between 4-6. Exemplary emulsions for use in the compositions of the invention are set forth below.

### Deodorant Properties and Agents

The personal care compositions of the present invention include at least one agent that exhibits deodorant and/or sweat proof activity. As used herein, “deodorant” refers to compositions that are effective in directly or indirectly reducing unwanted body odors associated with perspiration and/or bacteria on the surface of the skin. “Deodorants” may reduce odor through a variety of means, for example, by suppression of bacterial activity, antimicrobial mechanisms, chemical interference with odor generation mechanisms, removal or modification of feedstuff for odor-producing bacteria, and the like. A deodorant may function as an antiperspirant in reducing the production of perspiration, for example, by decreasing the size of pores associated with sweat glands, blocking sweat glands, reducing the flow of blood or other fluids to sweat glands, and the like, but need not do so to be considered a deodorant.

The deodorant properties of the present invention stem, at least in part, from the incorporation of an effective amount of at least one of sodium bicarbonate (baking soda), cornstarch, kaolin clay or bentonite clay. In one embodiment, the composition includes sodium bicarbonate or cornstarch in an amount effective to reduce body odor. In another embodiment, the composition includes sodium bicarbonate and cornstarch in amounts effective to reduce body odor. In yet another embodiment, the composition includes kaolin clay or bentonite clay in an amount effective to reduce body odor. In yet a further embodiment, the composition includes kaolin clay and bentonite clay in amounts effective to reduce body odor.

In particular embodiments, the deodorant agent is present at about 5% to about 35%, from about 10% to about 30% or about 5% to about 15% by weight of the composition.

In particular embodiments, particularly where the composition includes sodium bicarbonate, the composition further includes Maranta arundinacea (arrowroot). The arrowroot is present in an amount to absorb moisture, neutralize negative reactions of sodium bicarbonate, exhibit anti-fungal properties and/or exhibit anti-bacterial properties.

### Other Elements including Concealer Properties and Agents

The personal care compositions of the present invention include at least one agent that exhibits concealer properties. As used herein, the term “concealer” and “concealer composition,” refer to a cosmetic composition that is applied to skin to hide a perceived imperfection, for example, discoloration such as hyper pigmentation (e.g., age spot, mole, freckle and the like), scar, tattoo, fine line, wrinkle, and vascularization. As used herein, “hyper-pigmentation” and variations thereof mean an area of skin that appears darker than the natural tone of the surrounding skin (i.e., without makeup) due to an increase in melanin production. Nonlimiting examples of hyper-pigmentation include freckles, moles, birthmarks, solar lentigines (age spots or liver spots), melasma or chloasma. “Hypo-pigmentation” and variations thereof mean an area of skin that appears lighter than the natural tone of the surrounding skin due to a decrease in melanin production. By way of example, the personal care concealer compositions of the present invention can be used to conceal discoloration, such as that caused by hyper-pigmentation and hypo-pigmentation, in addition to other imperfections, such as scars, pores, etc.

### (i) Spreadability Agent

In a particular embodiment, the personal care concealer compositions of the invention include an agent that enhances spreadability and consistency of the composition. In a particular embodiment, the composition includes isododecane. Alternatively, or in combination, the personal care composition includes trisiloxane, cyclopentasiloxane or a trisiloxane blend, which serve to enhance spreadability and consistency of the composition. While serving the aforementioned function, this agent and, in particular, isododecane, further serves to inhibit water evaporation from the skin and render the composition substantially non-transferable.

The spreadability agent is preferably volatile in nature. As such, the agent evaporates, leaving the product behind. In this regard, the product is imbued with a creamy feel. An exemplary agent that exhibits such volatile properties is isododecane. Alternative volatile agents for use in the present invention include oil based hydrocarbons or cyclomethicones.

Given its ability to imbue the product with a creamy feel, to enhance the spreadability and consistency of the composition, to inhibit water evaporation from the skin, and to contribute to the non-transferable nature of the composition, isododecane is a preferable additive to the personal concealer compositions of the invention.

The spreadability agent and, in particular, isododecane, is present in an amount effective to imbue the composition with sufficient spreadability and nontransferability. In a particular embodiment, the spreadability agent, for example, isododecane, may be present at about 30% to about 60%, at about 35% to about 55% or about 40% to about 50% by weight of the composition.

### (ii) Water Resistant Agent/Film Former

In certain embodiments, the personal care concealer compositions of the invention additionally include a water resistant agent. For example, the water resistant agent may include a silica polymer, for example, silica silylate, silica dimethyl silylate, trimethylsiloxysilicate, trifluoropropyldimethyl/trimethylsiloxysilicate, or a combination thereof. In a particular embodiment, the water resistant agent includes trimethylsiloxysilicate. Alternatively or in combination, the water resistant agent includes beeswax.

The water resistant agent, for example, trimethylsiloxysilicate and/or beeswax, is present in an amount effective to hold pigments in place and imbue the composition with sufficient water resistance.

In a particular embodiment, the water resistant agent, for example, trimethylsiloxysilicate and/or beeswax, may be present at about 5% to about 40%, about 10% to about 30% or about 10% to about 20% by weight of the composition.

In certain embodiments, the personal care concealer compositions of the invention additionally include a film former or include a water resistant agent that serves as a film former. The film former is important to minimize or eliminate the transfer of pigment/color, for example, to clothing. Through incorporation of the film former, the concealer composition binds to the skin and does not transfer to clothing.

The film former may be selected from the group consisting of trimethylsiloxysilicate, a polymethylsilsesquioxane, a tosylamide/formaldehyde Resin, a polyester resin, a trimellitic anhydride monomer, a vinyl caprolactam/VP/dimethylaminoethyl methacrylate copolymer, isododecane and ethylene/propylene copolymer, styrene/butadiene copolymer, polyvinylpyrrolidone (PVP), a PVP and VP/eicosene copolymer, triacontanyl PVP, a styrene/acrylates copolymer, a VP/Eicosene Copolymer, an acrylates copolymers, an acrylates/C12-22 alkyl methacrylate copolymer, VP/Dimethiconylacrylate/Polycarbamyl Polyglycol Ester (and) VP/Polycarbamyl Polyglycol Ester, VP/Polycarbamyl, Adipic Acid/Neopentyl Glycol/Trimellitic Anhydride Copolymer, Polyurethane-18,48 and alike, Polyester Resins, a Vinyl Caprolactam/VP/Dimethylaminoethyl Methacrylate copolymer, Maltose (and) Agave Tequilana Leaf Extract, an Ethylene/Propylene copolymer, a styrene/butadiene copolymer, a soyamine/IPDI copolymer, trifluoropropyldimethylsiloxy/trimethylsiloxy Silsesquioxane (and) dimethicone, and a combination thereof. In a particular embodiment, the film former is trimethylsiloxysilicate. In another embodiment, the film former is a styrene/acrylates copolymer.

In a particular embodiment, film former, for example, trimethylsiloxysilicate or a styrene/acrylates copolymer, may be present at about 1% to about 25%, about 5% to about 30% or about 10% to about 20% by weight of the composition.

(iii) Thickening Agent

In certain embodiments, the personal care concealer compositions of the invention additionally include a thickening agent. Thickening may be achieved by waxes or montmorillonite minerals, or various types of associative thickeners.

Suitable waxes include animal, vegetable, mineral, and synthetic waxes, or silicone waxes. Generally such waxes have a melting point ranging from about 28 to 125° C., preferably about 30 to 100° C. Examples of waxes include acacia, beeswax, ceresin, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG 6-20 beeswax, or PEG-12 carnauba wax.

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone. Such silicones include, but are not limited to stearoxydimethicone, behenoxy dimethicone, stearyl dimethicone, cetearyl dimethicone, and so on.

Suitable waxes are also set forth in U.S. Pat. No. 5,725,845 which is hereby incorporated by reference in its entirety.

If present, suitable montmorillonite minerals include natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, and bentones, either alone or in combination with carbonate activators such as propylene carbonate.

Other types of thickening agents include fatty acids or alcohols, optionally substituted with hydroxyl groups, for example 12-hydroxystearic acid. Such fatty acids or alcohols have the general formula R—COOH or R—OH respectively, where R is a straight or branched chain, saturated or unsaturated alkyl having from about 6 to 45 carbon atoms, wherein one or more of the alkyl groups may be substituted with functional groups such as hydroxy, alkoxy, alkyl, and so on.

In a particular embodiment, the personal care concealer compositions of the invention include disteardimoium hectorite as a thickening agent. Alternatively, or in combination, the personal care concealer compositions of the invention include bentonite.

The thickening agent, for example, disteardimoium hectorite and/or bentonite, may be present at about 1% to about 25%, about 2% to about 20% or about 3% to about 15% by weight of the composition.

### (iv) Coloring Agents/Pigments

In certain embodiments, the personal care concealer compositions of the invention additionally include a coloring agent. The composition may include coloring agents such as dyes, pigments, lakes and toners. As used herein, the term “pigments” embraces lakes and fillers such as talc, calcium carbonate, etc. Exemplary inorganic pigments include, but are not limited to, inorganic oxides and hydroxides such as magnesium oxide, magnesium hydroxide, calcium oxide, calcium hydroxides, aluminum oxide, aluminum hydroxide, iron oxides (a-Fe203, y-Fe203, Fe304, FeO) and iron hydroxides including red iron oxide, yellow iron oxide and black iron oxide, titanium dioxide, titanium lower oxides, zirconium oxides, chromium oxides, chromium hydroxides, manganese oxides, manganese hydroxides, cobalt oxides, cobalt hydroxides, cerium oxides, cerium hydroxides, nickel oxides, nickel hydroxides, zinc oxides and zinc hydroxides and composite oxides and composite hydroxides such as iron titanate, cobalt titanate and cobalt aluminate and the like. Preferably, the inorganic oxide particles may be selected from silica, alumina, zinc oxide, iron oxide and titanium dioxide particles, and mixtures thereof.

In various embodiments, the personal care concealer composition includes at least one of titanium dioxide, yellow iron oxide, red iron oxide or black iron oxide. In one embodiment, the personal care concealer composition includes each of titanium dioxide, yellow iron oxide, red iron oxide or black iron oxide.

In one embodiment, the pigments have a particle size from 5 nm to 500 microns, or from 5 nm to 250 microns, or from 10 nm to 100 microns. In some embodiments, the particle size (median) will be less than bout 5 microns or less than 1 micron.

The colorant may be present at about 0.1% to about 15%, about 0.5% to about 15%, about 0.5% to about 10%, about 0.5% to about 5% or about 1% to about 3% by weight of the composition.

In certain embodiments, the personal care concealer compositions of the invention, the composition includes an opacifying agent. Opacifying agents can serve to impart a rich, creamy feel to the cosmetic formulation. Opacifying agents serve to prevent light transmission and as such, conceal the presence of discoloration, imperfections and skin defects. In certain embodiments, the opacifying agents are pigments that also exhibit opacifying affects. For example, the compositions may include at least one of yellow iron oxide, red iron oxide or black iron oxide.

### (v) Emulsifiers

In certain aspects of the present invention, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

The emulsifier may be present at about 0.1% to about 15%, about 0.5% to about 15%, about 0.5% to about 10%, about 0.5% to about 5% or about 1% to about 3% by weight of the composition.

### (vi) Preservative

In certain embodiments, the personal care concealer compositions of the invention additionally include a preservative. Suitable preservatives for compositions of this invention include, but are not limited to, alkyl esters of para-hydroxybenzoic acid, hydantoin derivatives such as 1,3-bis (hydroxymethyl)-5,5-dimthylhydantoin, propionate salts, and a variety of quaternary ammonium compounds such as benzalkonium chloride, quaternium 15 DOWICIL™ 200, benzethonium Chloride, and methylbenzethonium chloride. Additional preservatives include disodium EDTA, phenoxyethanol, methyl paraben, propyl paraben, imidazolidinyl urea (commercially available as GERMALL® 115), sodium dehydroacetate and benzyl alcohol.

In a particular embodiment, the preservative includes propylparaben. In an alternative embodiment, the preservative includes benzyl alcohol.

Preservatives preferably are employed in amounts ranging from about 0.01% to about 5%, about 0.05% to about 3% or about 0.1% to about 1% by weight of the composition.

(vii) Hyper-Pigmentation Agent

In certain embodiments, the personal care concealer compositions of the invention additionally include a hyper-pigmentation agent. In this regard, the compositions of the invention can serve, not merely to conceal, but to treat hyper-pigmentation and discoloration of the skin. Such agents serve to reduce the visible hyper-pigmentation and discoloration over time. For example, the composition of the invention may include mulberry (e.g., mulberry extract), licorice, kojic acid, Vitamin B3 (niacinamide), arbutin or a combination thereof.

(viii) Brightening Agent

In certain embodiments, the personal care concealer compositions of the invention additionally include a brightening agent (also known as a “brightener” or “skin lightener”). Brightening agents can serve, for example, to lighten skin and/or reduce the appearance of skin pigmentation over time (e.g., by reducing the production of melanin in the skin). In some embodiments, the skin pigmentation is hyper-pigmentation. Additional agents, e.g., bleaching agents, peeling agents, and/or depigmenting agents, can also be included in the personal care concealer compositions of the invention. As well understood by those skilled in the art, these additional agents may lighten skin and/or reduce the appearance of skin pigmentation over time via a variety of different mechanisms. As used herein, the term “brightening agent” encompasses all such agents, of both similar and different mechanisms of action, that can be used to achieve a skin lightening effect.

Exemplary brightening agents include, but are not limited to, licorice, kojic acid, arbutrin, mulberry (e.g., mulberry extract), Vitamin B3 (niacinamide), Vitamin C, citrus extracts, willow bark extract, lactic acid, Aloe vera, bearberry extract, Phyllanthus emblica, ALPAFLOR® GIGAWHITE™ (INCI name: Water, Glycerin, Alcohol, Malva sylvestris (Mallow) Extract, Mentha piperita (Peppermint) Leaf Extract, Primula Veris Extract, Alchemilla Vulgaris Extract, Veronica Officinalis Extract, Melissa Officinalis Leaf Extract, Achillea Millefolium Extract), glycolic acid, phenylethyl resorcinol, bisabolol, butyl methoxydibenzoylmethane, 4-butylresorcinol, Pelvetia canaliculata extract, synthetic fluorphlogopite, titanium dioxide, tin oxide, Hexapeptide-2, Poncirus trifoliata fruit extract, 1-Methylhydantoin-2-Imide, Bambusa arundinacea stem extract, Equisetum fluviatile extract, Camellia Japonica flower extract, Pinus densiflora leaf extract, Terminalia ferdinandiana fruit extract, Carica papaya (papaya) fruit extract, Arctostaphylos uva-ursi leaf extract, hydroquinone, mandelic acid, or a combination thereof.

In some embodiments, certain agents, for example, mulberry (e.g., mulberry extract), licorice, kojic acid, Vitamin B3 (niacinamide), arbutin or a combination thereof can serve as both a hyper-pigmentation treatment agent and a brightening agent.

In some embodiments, the brightening agent is a bleaching agent (e.g., hydroquinone). The exemplary bleaching agent hydroquinone functions as a depigmenting agent that suppresses melanocyte metabolic processes of the skin, e.g., by inhibiting the enzymatic oxidation of tyrosine to DOPA (3, 4-dihydroxyphenylalanine).

In some embodiments, the brightening agent is a peeling agent (e.g., fruit acids, such as glycolic acid, mandelic acid, kojic acid, and/or salicylic acid). Without wishing to be bound by any particular theory, a peeling agent is capable of exfoliating and/or removing one or more layers of the skin, thereby decreasing pigmentation. Additional peeling agents are also disclosed, for example, in U.S. Pat. No. 5,505,948, U.S. Pat. No. 4,608,370, and U.S. Pat. No. 5,164,413, the entire contents of which are incorporated herein by reference.

In certain embodiments, the brightening agent is both a bleaching agent and a peeling agent (e.g., fruit acids, such as glycolic acid, mandelic acid, kojic acid, and salicylic acid).

In some embodiments, the brightening agent is capable of inhibiting melanin synthesis.

In some embodiments, the brightening agent has anti-tyrosinase activity. Tyrosinase enzyme has been shown to be involved in melanin synthesis and catalyzes two different reactions: the hydroxylation of monophenols to o-diphenols (monophenolase activity) and the oxidation of o-diphenols to o-quinones (diphenolase activity). In the cell, o-quinone is then transformed into melanin via a series of non-enzymatic reactions (Muddathir A M, et al. (2017) S African J Botany. 109:9-15). Without wishing to be bound by any particular theory, a brightening agent with anti-tyrosinase activity can inhibit both tyrosinase enzyme activity and melanin production.

In various embodiments, the personal care concealer composition includes at least one brightening agent as described herein. In various embodiments, the personal care concealer composition includes at least two brightening agent as described herein. In various embodiments, the personal care concealer composition includes at least three brightening agent as described herein.

The brightening agent may be present in an amount effective to lighten skin and/or reduce the appearance of skin pigmentation, e.g., by reducing and/or inhibiting the synthesis of melanin in the skin. In some embodiments, the brightening agent may be present in an amount effective to reduce and/or inhibit tyrosinase enzyme activity. In some embodiments, the brightening agent may be present at about 0.001% to about 30%, about 0.001% to about 25%, about 0.01% to about 20%, about 0.1% to about 15%, about 0.5% to about 15%, about 0.5% to about 10%, about 0.5% to about 5% or about 1% to about 3% by weight of the composition. The amount of brightening agent varies, at least in part, based on the nature of the agent.

### Fragrance

In certain embodiments, the personal care concealer compositions of the invention additionally include a fragrance agent. Preferably, the fragrance agent is a natural fragrance powder. Without wishing to be bound by any particular theory, the use of a natural fragrance powder, which is unconventional, contributes to the non-transferability properties of the composition.

In a particular embodiment, the fragrance agent has a scent selected from the group consisting of tea tree, lavender, lemongrass, citrus, rosemary, and a combination thereof.

Alternatively, the fragrance agent may be a perfume oil or an essential oil. For example, suitable perfume oils can contain natural fragrance mixtures, such as are obtainable from plant or animal sources, for example pine, citrus, jasmine, rose, lily or ylang-ylang oil. Low-volatility essential oils, which are mostly used as aroma components, are also suitable as perfume oils, for example sage oil, chamomile oil, melissa oil, mint oil, cinnamon leaf oil, lime blossom oil, juniper berry oil, vetiver oil, olibanum oil, galbanum oil, laudanum oil, clove oil, iso-eugenol, thyme oil, bergamot oil, geranium oil and rose oil.

The fragrance agent, e.g., natural fragrance powder, may be present at about 0.1% to about 5%, about 0.2% to about 5%, about 0.2% to about 1% by weight of the composition.

In a particular embodiment, the personal care composition includes a fragrance agent that is designed to release fragrance at some predetermined time, for example, 4, 6, 8, 10 or 12 hours after administration. Alternatively, or in combination, the personal care concealer composition includes a fragrance agent that is designed to release upon contact with sweat.

The compositions of the invention may further include a fragrance absorption agent, which serves to enhance the ability of the scent to be held and absorbed. In a particular embodiment, the fragrance absorption agent includes dextrose.

The fragrance absorption agent, e.g., dextrose, may be present at about 0.1% to about 5%, about 0.2% to about 5%, or about 0.2% to about 1% by weight of the composition.

### Application Device

In one aspect, the present invention is directed to a cosmetic product including a device for holding and applying the personal care concealer compositions of the invention. In a preferable embodiment, the device includes a base for holding the personal care concealer product. In a particular embodiment, the device further includes an applicator for applying the composition to the body, for example, the underarm. For example, the applicator may include a sponge or sponge-tipped applicator, a swab (for example, a cotton-tipped swab), a foam, a brush, a wipe, and combinations thereof. In a particular embodiment, the applicator is a foam or a sponge. Preferably, the applicator is a sponge.

The device is designed such that the composition, stored in the base, may be transferred to the applicator from the base. For example, the composition may be transferred to the applicator by squeezing, a pump or by other suitable means. The composition is then applied to the body by contacting the applicator, for example, the sponge, to the skin, for example, the underarm. Contact may include rolling, light pressure, dabbing, rubbing, wiping, or any other suitable means. Subsequently, the composition can be dabbed and blended under the arm to give a more natural look using the applicator, e.g., sponge.

The compositions of the present may also be designed for delivery in an air brushing system or as an aerosol spray.

Alternatively, the compositions of the invention may be designed for delivery as an airbrush, an airtight stick, via silicon or sponge.

### Other Uses

The compositions of the present invention are not limited to use in the underarm area. The compositions may be applied to any other parts of the body affected by skin discoloration, imperfections, redness, blemishes, darkening, acne, scarring, birthmarks, melasma, age spots, lentigines, sun exposure, and/or body odor. For example, the compositions may be applied to other parts of the body affected by hyperpigmentation and/or Acanthosis Nigricans conditions. In some embodiments, the compositions may be applied to conceal skin discoloration and/or imperfections, e.g., on the face, back, and/or chest. The compositions may also be applied to conceal tattoos. For example, the compositions may be applied to other regions of the body, for example, the groin, bikini line, skin folds, feet, back, face, chest, etc.

In order that the invention described herein may be more fully understood, the following examples are set forth. It should be understood that these examples are for illustrative purposes only and are not to be construed as limiting this invention in any manner.

All features of each of the aspects of the invention apply to all other aspects mutatis mutandis. The contents of all references, patents, pending patent applications and published patents, cited throughout this application are hereby expressly incorporated by reference.

### EXAMPLES

### Example 1

A first formulation is prepared including:
1. Sodium Bicarbonate (Baking Soda) and/or Cornstarch—Effectively neutralizes odor causing bacteria, absorbs sweat
2. Maranta arundinacea (Arrowroot)—Helps absorb moisture and neutralizes any negative reactions of Baking Soda; Also has anti-fungal and anti-bacteria properties
3. Blend of various natural Fragrance Powders (Tea Tree, Lavender, Lemongrass, Citrus, Rosemary etc.)—For long lasting fresh scent and has antibacterial and antimicrobial properties that kill bacteria that causes unpleasant odor)
4. Dextrose—Holds and absorbs the scent of fragrance
5. Isododecane—Enhances spreadability and consistency; helps with evaporation of water from the skin making it non-transferrable
6. Trimethylsiloxysilicate—Helps hold pigments in place while providing water resistance
7. Disteardimoium Hectorite—Thickening agent used in cosmetics and deodorants
8. Propylparaben—Preservative used to prevent the growth of harmful bacteria and mold.

### Example 2

A second formulation is prepared including:
1. Bentonite Clay—Absorbs moisture and acts as a detoxification agent
2. Blend of various natural Fragrance Powders (Tea Tree, Lavender, Lemongrass, Citrus, Rosemary etc.)—For long lasting fresh scent and has antibacterial and antimicrobial properties that kill bacteria that causes unpleasant odor)
3. Dextrose—Holds and absorbs the scent of fragrance
4. Isododecane—Enhances spreadability and consistency; helps with evaporation of water from the skin making it non-transferrable
5. Trimethylsiloxysilicate—Helps hold pigments in place while providing water resistance
6. Disteardimoium Hectorite—Thickening agent used in cosmetics and deodorants
7. Propylparaben—Preservative used to prevent the growth of harmful bacteria and mold.

### Example 3

A third formulation is prepared including:
1. Kaolin Clay—Absorbs moisture and acts as a detoxification agent
2. Blend of various natural Fragrance Powders (Tea Tree, Lavender, Lemongrass, Citrus, Rosemary etc.)—For long lasting fresh scent and has antibacterial and antimicrobial properties that kill bacteria that causes unpleasant odor)
3. Dextrose—Holds and absorbs the scent of fragrance
4. Isododecane—Enhances spreadability and consistency; helps with evaporation of water from the skin making it non-transferrable
5. Trimethylsiloxysilicate—Helps hold pigments in place while providing water resistance
6. Disteardimoium Hectorite—Thickening agent used in cosmetics and deodorants
7. Propylparaben—Preservative used to prevent the growth of harmful bacteria and mold.

### Example 4

A fourth formulation is prepared including:
1. Sodium Bicarbonate (Baking Soda) and/or Cornstarch—Effectively neutralizes odor causing bacteria, absorbs sweat
2. Maranta Arundinacea (Arrowroot)—Helps absorb moisture and neutralizes any negative reactions of Baking Soda; Also has anti-fungal and anti-bacteria properties
3. Blend of various natural Fragrance Powders (Tea Tree, Lavender, Lemongrass, Citrus, Rosemary etc.)—For long lasting fresh scent and has antibacterial and antimicrobial properties that kill bacteria that causes unpleasant odor)
4. Dextrose—Holds and absorbs the scent of fragrance
5. Isododecane—Enhances spreadability and consistency; helps with evaporation of water from the skin making it non-transferrable
6. Beeswax—Provides water resistance
7. Bentonite—Thickening agent used in cosmetics and deodorants
8. Benzyl Alcohol—Preservative used to prevent the growth of harmful bacteria and mold.

### Example 5

A fifth formulation is prepared including:
1. Bentonite Clay—Absorbs moisture and acts as a detoxification agent
2. Blend of various natural Fragrance Powders (Tea Tree, Lavender, Lemongrass, Citrus, Rosemary etc.)—For long lasting fresh scent and has antibacterial and antimicrobial properties that kill bacteria that causes unpleasant odor)
3. Dextrose—Holds and absorbs the scent of fragrance
4. Isododecane—Enhances spreadability and consistency; helps with evaporation of water from the skin making it non-transferrable
5. Beeswax—Provides water resistance
6. Bentonite—Thickening agent used in cosmetics and deodorants
7. Benzyl Alcohol—Preservative used to prevent the growth of harmful bacteria and mold.

### Example 6

A sixth formulation is prepared including:
1. Kaolin Clay—Absorbs moisture and acts as a detoxification agent
2. Blend of various natural Fragrance Powders (Tea Tree, Lavender, Lemongrass, Citrus, Rosemary etc.)—For long lasting fresh scent and has antibacterial and antimicrobial properties that kill bacteria that causes unpleasant odor)
3. Dextrose—Holds and absorbs the scent of fragrance
4. Isododecane—Enhances spreadability and consistency; helps with evaporation of water from the skin making it non-transferrable
5. Beeswax—Provides water resistance
6. Bentonite—Thickening agent used in cosmetics and deodorants
7. Benzyl Alcohol—Preservative used to prevent the growth of harmful bacteria and mold.

### Example 7

A seventh formulation was prepared including:
1. Isododecane: Emollient and solvent that enhances spreadability and helps prevent evaporation of water from the skin
2. Trimethylsiloxysilicate: Transfer and wash-off resistance combined with a flexible film for long-lasting, long-wearing, smudge-proof functionality
3. Isododecane Disteardimonium Hectorite: Provides a water-proof finish
4. Tribehenin: Provides products with a smooth application, thickening agent
5. Dimethicone: Emollient used for silky feel and reducing greasiness
6. Trihydroxystearin: Thickener for oils
7. Sodium Bicarbonate: Odor Protection
8. Tapioca starch: Absorbs moisture
9. Tea Tree Oil: Antiseptic and Antibacterial, Kills Odor
10. Citrus Oil: Scent, Fragrance, Antimicrobial
11. Natural Oil Blend consists of [Aroma (natural vanilla), Mentha Virdis (Spearmint) Leaf Oil, Mentha Virdis (Spearmint) Leaf Oil, Gurjun Balsam Oil, Cananga Odorata (Ylang Ylang) Flower Oil, Cinnamomum Camphora L. (Ho) Leaf Oil, Cistus ladaniferusresin, Citrus Limon (Lemon) Peel Extract, Cymbopogon Martini (Palmarosa) Oil, Lavandula Hybrida (Lavandin) Oil, Ricinus Communis (Castor) Seed Oil, Pelargonium graveolens flower oil, Pogostemon Cablin (Patchouli) Oil, Juniperus Mexicana Oil, Cananga Odorata var Macrophylla (Cananga) oil, Artemisia Pallens(Davana) Oil, Anthemis Nobilis (Chamomile) Flower Oil, Gaultheria Precumbens (Wintergreen) Leaf oil, Myroxylon Pereira (Balsam Peru) Oil, Myroxylon Toleuferum (Tolu Balsam) Oil]: For Scent and Odor Protection
12. Titanium Dioxide: pigment/sun screen/actives
13. Yellow Iron Oxide: pigment/opacifying agent
14. Red Iron Oxide: pigment/opacifying agent
15. Black Iron Oxide: colorant/pigment/opacifying agent

### Example 8

The personal care concealer composition as described in Example 7 was tested on several subjects having varying physical attributes as follows:
Light, medium and dark skin tones
Varying levels of underarm discoloration: mild, moderate, severe
Varying levels of sweating: light, normal, excessive

Subjects applied the composition to assess the ability of the product to exhibit deodorant, concealer and color correcting function. Specifically, feedback was requested on the following elements:
Coverage
Non-transferability
Texture
Long-lasting odor protection
   In order to assess these functions, the subjects were requested to:
Put on clothes a few minutes after the product had dried to ensure non-transferability
Remove clothes post application to ensure non-transferability
Blot and wipe tissue over underarm post application to ensure non-transferability
Apply composition on one underarm and no product on the other underarm in the morning post showering
Throughout the day, conduct normal daily routine activities including walking, working, running errands
Test for odor within the following scenarios to validate physical activity induced, and stress induced types of sweat:
Speed walk on a weekend during which temperatures routinely exceeded 90° F.
Undergo stressful conditions including preparing for critical client presentations under compressed timelines and undertaking a heavy workload

Under both normal and extreme scenarios, as described above, all subjects reported as follows:
They were pleased with the level of coverage
They were pleased with the ability to build up the product if needed
The product did not transfer after it set
They did not smell through the full day within the underarm to which the composition had been applied
The texture felt creamy and non-irritating
The scent was fresh and pleasing

Of note, amongst the group of 10 subjects reporting such functionality, one subject self identifies as easily getting stressed and anxious as a general matter.

Moreover, success of the product in terms of concealing and correcting color across varying levels of underarm discoloration severity, including severe cases of discoloration, was reported.

### Example 9

An eighth water based emulsion formulation was prepared as follows:
1. Titanium Dioxide: pigment/sun screen/actives
2. Yellow Iron Oxide: pigment/opacifying agent
3. Red Iron Oxide: pigment/opacifying agent
4. Black Iron Oxide: colorant/pigment/opacifying agent
5. Water (Aqua): moisturizing
6. Styrene/Acrylates Copolymer: film forming/water proofing
7. C12-1C15 Alkyl Benzoate: emollient
8. Caprylic/Capric Triglyceride: emollient/binding
9. Butylene Glycol: humecant/emulsion stability/solvent/conditioner
10. Cetearyl Alcohol (and) Polysorbate 60: emulsifying/viscosity controlling
11. Propylene Glycol Stearate: Emulsifier/Emulsifier non-GMO sunflower derived
12. Sodium bicarbonate: odor protection
13. Tapioca Starch: absorbs moisture
14. Gluconolactone (and) Sodium Benzoate: preservative/odor masking/pH control
15. Cellulose Gum: Stabilizing
16. Fragrance (Citrus): Odor masking/preservative/natural
17. Disodium EDTA: Chelating agent
18. Ylang & lavender dreams (MOD: Odor masking
19. Melaleuca Alternifolia (tea tree) leaf oil: antiseptic and antibacterial, kills odor

The deodorizing components in both are sodium bicarbonate and tea tree oil as well as the ylang and lavender blend of essential oils. The film forming properties in both further aid in inhibiting perspiration to permeate through the skin thereby minimizing odor development. The evaporation of water in the outer phase of the emulsion, leaves behind the film as well as the non-volatile components of the oil phase (essential oils and all of the rest of the solids and oils.

The composition may optionally include magnesium aluminum silicate to assist in viscosity control and/or to serve as a suspending agent. Preferably, however, the composition is formulated as an aluminum free composition in which the magnesium aluminum silicate is replaced with at least one of silver chloride, titanium dioxide and zinc citrate.

### Example 10

The personal care concealer composition as described in Example 9 was tested on an individual. The composition was tested on 3 individuals representing the following attributes:
Light, medium, dark skin tones
Varying levels of underarm discoloration: mild, moderate, severe
Varying levels of sweating: light, normal, excessive

The product was found to exhibit the following properties:
The product did not transfer after it set (1-2 minutes)
   Put on clothes a few minutes after the product had dried to ensure non-transferability
   Removed clothes post application to ensure non-transferability
   Blotted and wiped tissue over underarm post application to ensure non-transferability
Level of coverage and ability to build up the product to conceal discoloration and imperfections was sufficient
Underarm did not smell most of the day.
Scent was fresh and pleasing
Formulation was non-irritating.

1.-45. (canceled)

46. A personal care composition that reduces body odor comprising
(i) a deodorant agent in an amount effective to reduce sweat and/or body odor; and
(ii) a film former.

47. The personal care composition of claim 46, wherein the composition absorbs sweat;
wherein the composition conceals skin discoloration, imperfections, dark spots, large pores or darkening;
wherein the composition serves to color correct;
wherein the composition lightens skin and/or reduces the appearance of skin pigmentation; and/or
wherein the composition is substantially non-transferable.

48.-50. (canceled)

51. The personal care composition of claim 46, further comprising a spreadability agent.

52. The personal care composition of claim 46, wherein the deodorant agent comprises at least one of sodium bicarbonate, arrowroot, cornstarch, bentonite clay, kaolin clay, activated charcoal powder, tapioca starch, and magnesium hydroxide in an amount effective to reduce sweat and/or body odor.

53. (canceled)

54. The personal care composition of claim 51, wherein the spreadability agent is selected from the group consisting of isododecane, trisiloxane, cyclopentasiloxane, a trisiloxane blend and a combination thereof.

55. The personal care composition of claim 46, wherein the film former is selected from the group consisting of trimethylsiloxysilicate, a polymethylsilsesquioxane, a tosylamide/formaldehyde Resin, a polyester resin, a trimellitic anhydride monomer, a vinyl caprolactam/VP/dimethylaminoethyl methacrylate copolymer, isododecane and ethylene/propylene copolymer, styrene/butadiene copolymer, polyvinylpyrrolidone (PVP), a PVP and VP/eicosene copolymer, triacontanyl PVP, a styrene/acrylates copolymer, a VP/Eicosene Copolymer, an acrylates copolymers, acrylates/octylacrylamide copolymer, an acrylates/C12-22 alkyl methacrylate copolymer, VP/Dimethiconylacrylate/Polycarbamyl Polyglycol Ester (and) VP/Polycarbamyl Polyglycol Ester, VP/Polycarbamyl, Adipic Acid/Neopentyl Glycol/Trimellitic Anhydride Copolymer, Polyurethane-18,48 and alike, Polyester Resins, a Vinyl Caprolactam/VP/Dimethylaminoethyl Methacrylate copolymer, Maltose (and) Agave Tequilana Leaf Extract, an Ethylene/Propylene copolymer, a styrene/butadiene copolymer, a soyamine/IPDI copolymer, trifluoropropyldimethylsiloxy/trimethylsiloxy Silsesquioxane (and) dimethicone.

56. The personal care composition of claim 46, wherein the personal care composition is an oil based composition, optionally, wherein the film former is selected from the group consisting of trimethylsiloxysilicate, a polymethylsilsesquioxane, a tosylamide/formaldehyde Resin, a polyester resin, a trimellitic anhydride monomer, a vinyl caprolactam/VP/dimethylaminoethyl methacrylate copolymer, isododecane and ethylene/propylene copolymer, styrene/butadiene copolymer, polyvinylpyrrolidone (PVP), a PVP and VP/eicosene copolymer, triacontanyl PVP and a combination thereof.

57.-58. (canceled)

59. The personal care composition of claim 46, wherein the personal care composition is a water based composition, optionally, wherein the film former is selected from the group consisting of an acrylates copolymer, an acrylates/octylacrylamide copolymer, a styrene/acrylates copolymer, polyvinyl alcohol (PVA), and a combination thereof.

60.-61. (canceled)

62. The personal care composition of claim 46, further comprising a pigment, optionally, wherein the pigment is selected from the group consisting of titanium dioxide, yellow iron oxide, red iron oxide, black iron oxide, and combinations thereof.

63. (canceled)

64. The personal care composition of claim 46, wherein the composition is an emulsion, optionally, wherein the emulsion is formed by stirring, high shear mixing, high-pressure homogenization or high-amplitude ultrasound (sonication).

65. The personal care composition of claim 64, further comprising an emulsifier, optionally, wherein the emulsifier is selected from the group consisting of esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, butylene glycol, cetearyl alcohol, propylene glycol stearate, and mixtures thereof.

66.-67. (canceled)

68. The personal care composition of claim 65, wherein the composition is water based and wherein the emulsifier comprises at least one of butylene glycol, cetearyl alcohol, polysorbate 60, propylene glycol stearate, and a combination thereof; and/or wherein the emulsifier has an HLB value between 8 and 18.

69. (canceled)

70. The personal care composition of claim 65, wherein the composition is an oil based emulsion, wherein water forms the inner phase of the emulsion, optionally, wherein the emulsifier has an HLB value between 4 and 6.

71.-72. (canceled)

73. The personal care composition of claim 64, wherein the composition is substantially free of an emulsifier.

74. The personal care composition of claim 46, wherein the composition is anhydrous.

75.-76. (canceled)

77. The personal care composition of claim 46, further comprising a hyper-pigmentation treatment agent, optionally, wherein the hyper-pigmentation treatment agent comprises mulberry, licorice, kojic acid, niacinamide, arbutin, tyrosine inhibitor or a combination thereof.

78. (canceled)

79. The personal care composition of claim 46, further comprising a brightening agent,
optionally, wherein the brightening agent is a bleaching agent, a peeling agent, and/or a fruit acid;
optionally, wherein the brightening agent inhibits tyrosinase activity and/or melanin synthesis; and/or
optionally, wherein the brightening agent comprises licorice, kojic acid, arbutrin, mulberry, Vitamin B3 (niacinamide), Vitamin C, citrus extracts, willow bark extract, lactic acid, Aloe vera, bearberry extract, Phyllanthus emblica, Gigawhite®, glycolic acid, phenylethyl resorcinol, bisabolol, butyl methoxydibenzoylmethane, Morus alba root extract, butylresorcinol, Pelvetia canaliculata extract, synthetic fluorphlogopite, titanium dioxide, tin oxide, Hexapeptide-2, Poncirus trifoliata fruit extract, 1-Methylhydantoin-2-Imide, Bambusa arundinacea stem extract, Equisetum fluviatile extract, Camellia Japonica flower extract, Pinus densiflora leaf extract, Terminalia ferdinandiana fruit extract, Carica papaya (papaya) fruit extract, Arctostaphylos uva-ursi leaf extract, hydroquinone, mandelic acid, salicylic acid, or a combination thereof.

80.-83. (canceled)

84. A cosmetic product comprising the personal care composition of claim 46 contained within a device, optionally, wherein the device further comprises an applicator for applying the composition to the body, a base which holds the personal care composition, a pump, an airbrush, and/or an aerosol spray.

85.-89. (canceled)

90. A method of treating a subject suffering from hyperpigmentation, comprising applying the personal care composition of claim 46 to a subject.

91.-92. (canceled)
